(19) United States
(12) Patent Application Publication
(10) Pub. No.: US 2004/0146882 A1
(43) Pub. Date: Jul. 29, 2004

(54) Human acid sensing ion channel 2b (hASIC2b), process for producing the same, and its use

(76) Inventors: Norikazu Gajya, Aichi-ken (JP); Katsuhiro Shinjo, Aichi-ken (JP); Kenji Taki, Aichi-ken (JP)

Correspondence Address: WARNER-LAMBERT COMPANY, 2800 PLYMOUTH RD, ANN ARBOR, MI, 48105, US

(21) Appl. No.: 10/639,720

(22) Filed: Aug. 12, 2003

Related U.S. Application Data
(60) Provisional application No. 60/402,992, filed on Aug. 12, 2002.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

A novel polynucleotide sequence, which encodes a novel polypeptide belonging to the proton (H⁺)-gated cation channel subfamily, human Acid Sensing Ion Channel 2b (hAISC2b), is provided. Since hASIC2b and the other hASICs seem to constitute at least part of the native proton-gated cation channel of nociceptive neurons, cells coexpressing hASIC2b and the other hASICs are useful for a method of screening candidate compounds modulating the perception of acidity with regard to nociception.

This United States utility application claims the benefit of U.S. Provisional application No. 60/402,992 filed on Aug. 12, 2002.

### FIELD OF THE INVENTION

The present invention relates to a novel polynucleotide sequence, which encodes a novel polypeptide belonging to the proton (H⁺)-gated cation channel subfamily, i.e., human Acid Sensing Ion Channel 2b (hAISC2b). The present invention also relates, inter alia, to processes of producing the polypeptide and its uses.

### BACKGROUND OF THE INVENTION

H⁺-gated cation channels are ligand-gated ion channels activated by protons. H⁺-gated cation channels with different pH sensitivities and kinetics were reported in sensory neurons, Bevan, S. & Yeats, J. J. Physiol. 433:145-161 (1991), Krishtal, O. A. & Pidoplichko, V. I. Brain Res. 214: 150-154 (1981), Akaike, N., Krishtal, O. A. & Maruyama, T. J. Neurophysiol. 63, 805-813 (1990), Kovalchuk, Yu, N., Krishtal, O. A. & Nowycky, M. C. Neurosci. Lett. 115: 237-242 (1990), Davies, N. W., Lux, H. D. & Morad, M. J. Physiol. 400, 159-187 (1988), Krishtal, O. A. & Pidoplichko, V. I. Neuroscience 6: 2599-2601 (1981), Akaike, N. & Ueno, S. Prog. Neurobiol. 43: 73-83 (1994), in neurons of the central nerve system (CNS), Akaike, N. & Ueno, S. Prog. Neurobiol. 43: 73-83 (1994), Ueno, S., Nakaye, T., & Akaike, N. J. Physiol. 447: 309-327 (1992), Grantyn, R., & Lux, H. D. Neurosci. Lett. 89: 198-203 (1988), and in oligodendrocytes, Sonthebier., H., Perquansky, M, Hoppe, D., Lux, H. D., Gratyn, R. & Kettenmann, H. J. Neurosci. Res. 24: 496-500 (1989), Waldmann, R., Champigny, G., Lingueglia, E., De Weille, J. R., Heurteaux, C. & Lazdunski, M. Annals New York Academy Of Sciences 67-76 (Apr. 30, 1999). The extracellular pH in tissue can decrease by more than two pH units during tissue acidosis, Reeh, P. W. & Steen, K. H. Prog. Brain. Res. 113: 143-151 (1996), Waldmann, R., Champigny, G., Lingueglia, E., De Weille, J. R., Heurteaux, C. & Lazdunski, M. Annals New York Academy Of Sciences 67-76 (Apr. 30, 1999), with inflammation and many ischemic conditions. It is believed that the sensation of pain accompanies a decrease in pH, Steen, K. H., Issberner, U. & Reeh, P. W. Neurosci. Lett 199: 29-32 (1995), Waldmann, R., Champigny, G., Lingueglia, E., De Weille, J. R., Heurteaux, C. & Lazdunski, M. Annals New York Academy Of Sciences 67-76 (Apr. 30, 1999). Thus, H⁺-gated cation channels in sensory nerve endings were proposed to be involved in the perception of pain with tissue acidosis, Bevan, S. & Yeats, J. J. Physiol. 433:145-161 (1991), Krishtal, O. A. & Pidoplichko, V. I. Neuroscience 6: 2599-2601 (1981), Reeh, P. W. & Steen, K. H. Prog. Brain. Res. 113: 143-151 (1996), Waldmann, R., Champigny, G., Lingueglia, E., De Weille, J. R., Heurteaux, C. & Lazdunski, M. Annals New York Academy Of Sciences 67-76 (Apr. 30, 1999).

The ASICs (Acid Sensing Ion Channels) are members of H⁺-gated cation channel subfamily belonging to the ENaC/DEG superfamily, Lad, C. C., Hong, K., Kryneli, M, Chalfie, M. & Driscoll, M. J. Cell. Biol. 133: 1071-1081 (1996), Renard, S., Lingueglia, E., Voilley, N., Lazdunski, M. & Barbry, P. J. Biol. Chem. 269, 12981-12986 (1994). The superfamily includes the epithelial Na⁺ channel (ENaC), Canessa, C. M., Horisberger, J. D. & Rossier, B. C. Nature 361: 467-470 (1993), Lingueglia, E., Voilley, N., Waldmann, H., Lazdunski, M. & Barbry, P. Febs Lett. 318: 95-99 (1993), Canessa, C. M., Schild, L., Buell, G., Thorens, B., Gautschi, I., Horisberger, J. D. & Rossier, B. C. Nature 367: 463-467 (1994), Lingueglia, E., Renard, S., Waldmann, R., Voilley, N., Champigny, G., Plass, H., Lazdunski, M. & Barbry, P., J. Biol. Chem. 269: 13736-13739 (1994), Waldmann, R., Champigny, G., Bassilana, F., Voilley, N. & Lazdunski, M. J. Biol. Chem. 270: 27411-27414 (1995), Waldmann, R., Champigny, G., Lingueglia, E., De Weille, J. R., Heurteaux, C. & Lazdunski, M. Annals New York Academy Of Sciences 67-76 (Apr. 30, 1999), a family of proteins designated as degenerins (DEG), Chalfie, M. & Woeinsky, E. Nature 345:410-416 (1990), Driscoll, M. & Chalfie, M. Nature 349: 588-593 (1991), Huang, M. & Chalfie, M. Nature 367: 467-470 (1994), Tavernarakis, N., Shreffler, W., Wang, S. & Driscoll, M. Neuron 18: 107-119 (1997), Lad, C. C., Hong, K., Kryneli, M, Chalfie, M. & Driscoll, M. J. Cell. Biol. 133: 1071-1081 (1996), Waldmann, R., Champigny, G., Lingueglia, E., De Weille, J. R., Heurteaux, C. & Lazdunski, M. Annals New York Academy Of Sciences 67-76 (Apr. 30, 1999), and the FMRFamide-gated Na⁺ channel (FaNaC), Lingueglia, E., Champigny, G., Lazdunski, M. & Barbry, P. Nature 378: 730-733 (1995), Waldmann, R., Champigny, G., Lingueglia, E., De Weille, J. R., Heurteaux, C. & Lazdunski, M. Annals New York Academy Of Sciences 67-76 (Apr. 30, 1999).

The four rat H⁺-gated cation channel subunits (ASIC1-4) were cloned recently and will be briefly discussed below.

Acid Sensing Ion Channel 1 (ASIC1, often referred to as ASIC1a), Waldmann, R., Champigny, G., Bassilana, F., Heurteaux, C. & Lazdunski, M. Nature 386: 173-177 (1997), the first member of the H⁺-gated Na⁺ channel subfamily, is expressed in both brain and dorsal root ganglion cells (DRGs). It is activated by pH variations below pH 7. The presence of this channel throughout the brain suggests that H⁺ might play an essential role as a neurotransmitter or neuromodulator, Waldmann, R., Champigny, G., Lingueglia, E., De Weille, J. R., Heurteaux, C. & Lazdunski, M. Annals New York Academy Of Sciences 67-76 (Apr. 30, 1999). Like other members of the ENaC/DEG superfamily, Lad, C. C., Hong, K., Kryneli, M, Chalfie, M. & Driscoll, M. J. Cell. Biol. 133: 1071-1081 (1996), Renard, S., Lingueglia, E., Voilley, N., Lazdunski, M. & Barbry, P. J. Biol. Chem. 269, 12981-12986 (1994), ASIC1 has two transmembrane domains with a large extracellular loop protein component, Waldmann, R., Champigny, G., Bassilana, F., Heurteaux, C. & Lazdunski, M. Nature 386: 173-177 (1997). Like the FaNaC channel, it seems to assemble as a tetramer, Coscoy, S., Lingueglia, E., Lazdunski, M. & Barbry, P. J. Biol. Chem. 273: 8317-8322 (1998). ASIC1 is permeable to not only Na⁺ and Li⁺ but also Ca²⁺, and desensitizes rapidly with a single exponential time course Waldmann, R., Champigny, G., Lingueglia, E., De Weille, J. R., Heurteaux, C. & Lazdunski, M. Annals New York Academy Of Sciences 67-76 (Apr. 30, 1999). ASIC1 is blocked by amiloride and its derivatives, benzamil and ethylisopropylamiloride, Waldmann, R., Champigny, G., Lingueglia, E., De Weille, J. R., Heurteaux, C. & Lazdunski, M. Annals New York Academy Of Sciences 67-76 (Apr. 30, 1999). The transcript encoding ASIC1 is alternatively spliced, which generates an additional derivative of the ASIC1 protein (referred to as ASIC1b), Waldmann, R., Champigny, G., Bassilana, F., Heurteaux, C. & Lazdunski, M. Nature 386: 173-177 (1997), Waldmann, R., Champigny, G., Lingueglia, E., De Weille, J. R., Heurteaux, C. & Lazdunski, M. Annals New York Academy Of Sciences 67-76 (Apr. 30, 1999). Both of the rat and human ASIC1a proteins and ASIC1b proteins have been cloned. The amino acid sequence of the human ASIC1a (formerly referred to as BNaC2) has been identified in a human cDNA library, Garcia-Anoveros, J., Derfler, B., Neville-Golden, J., Hyman, B. T. & Corey, D. P. Proc. Natl. Acad. Sci. USA 94: 1459-1464 (1997). WO 00/08149 discloses that the rat ASIC1a and human ASIC1a proteins are considered to be functionally equivalent. The amino acid sequences of these two proteins are highly homologous, but are not identical. Substitutions can readily be introduced within the primary sequence of the ASIC1a proteins without influencing their essential functional characteristics.

ASIC2a, mammalian neuronal degenerin homologues was in fact cloned before ASIC1a and previously named MDEG1, Waldmann, R., Champigny, G., Voilley, N., Lauritzen, I. & Lazdunski, M. J. Biol. Chem. 271, 10433-10436 (1996), Waldmann, R., Champigny, G., Lingueglia, E., De Weille, J. R., Heurteaux, C. & Lazdunski, M. Annals New York Academy Of Sciences 67-76 (Apr. 30, 1999), (for mammalian degenerin) or BNC1, Price, M. P., Snyder, P. M. & Welsh, M. J. J. Biol. Chem. 271: 7879-7882 (1996), Waldmann, R., Champigny, G., Lingueglia, E., De Weille, J. R., Heurteaux, C. & Lazdunski, M. Annals New York Academy Of Sciences 67-76 (Apr. 30, 1999)(for brain Na⁺ channel 1). ASIC2a shares 67% sequence identity with ASIC1a, and it was demonstrated shortly after the cloning of ASIC1a that MDEG1 is also a H⁺-gated cation channel, Lingueglia, E., De Weille, J. R., Bassilana, F., Heurteaux, C., Sakai, H., Waldmann, R. & Lazdunski, M. J. Biol. Chem. 272: 29778-29783 (1997), Champigny, G., Voilley, R., Waldmann, R. & Lazdunski, M. J. Biol. Chem. 273: 15418-15422 (1998), Waldmann, R., Champigny, G., Lingueglia, E., De Weille, J. R., Heurteaux, C. & Lazdunski, M. Annals New York Academy Of Sciences 67-76 (Apr. 30, 1999). That is, cation transport by both ASIC1a and ASIC2a is sensitive to amiloride and regulated by acid. Biophysical properties of these two channels are, however, different in that ASIC2a channel requires more acidic pH values, i.e., pH values below pH 5.5 for activation, desensitizes slower than ASIC1a, and is selective for Na⁺ over Ca²⁺. The ASIC2a mRNA was detected in neurons of the CNS and sensory neurons. It has been shown that the rASIC2a channel is activated by the same mutations that cause neurodegeneration in C. elegans. This suggests that a gain of function of ASIC2a might be involved in human forms of neurodegeneration, Waldmann, R., Champigny, G., Voilley, N., Lauritzen, I. & Lazdunski, M. J. Biol. Chem. 271, 10433-10436 (1996). Both of the rat and human ASIC2a proteins have been cloned, Price, M. P., Snyder, P. M. & Welsh, M. J. J. Biol. Chem. 271: 7879-7882 (1996).

ASIC2b previously named MDEG2 is a splice variant of ASIC2a, Lingueglia, E., De Weille, J. R., Bassilana, F., Heurteaux, C., Sakai, H., Waldmann, R. & Lazdunski, M. J. Biol. Chem. 272: 29778-29783 (1997). From mouse and rat brain, ASIC2b has been cloned, which differs in the first 236 amino acids, including the first transmembrane region. This new membrane protein is expressed in both brain and sensory neurons. ASIC2b is activated neither by mutations that bring neurodegeneration once introduced in C. elegans degenerins nor by low pH. It can, however, associate both with ASIC2a and another recently cloned H⁺-gated channel DRASIC (hereinafter also referred to as ASIC3 and will be discussed below) to form heteropolymers that display different kinetics, pH dependencies, and ion selectivities. Of particular interest is the subunit combination specific for sensory neurons, ASIC2b/ASIC3 (MDEG2/DRASIC). This is because, in response to a drop in pH, the subunit combination gives rise to a biphasic current with a sustained current that discriminates poorly between Na⁺ and K⁺, like native H⁺-gated current recorded in dorsal root ganglion cells. This sustained current is thought to be required for the tonic sensation of pain caused by acids. WO 98/35034 discloses rat ASIC2b protein, Lingueglia, E., De Weille, J. R., Bassilana, F., Heurteaux, C., Sakai, H., Waldmann, R. & Lazdunski, M. J. Biol. Chem. 272: 29778-29783 (1997). Human ASIC2b protein, however, had not been cloned until the present invention was made.

ASIC3, that was previously named DRASIC (for DRG acid sensing ion channel)(ASIC3), is specifically present in DRGs, is absent in the brain, and displays biphasic kinetics, Krishtal, O. A., Osipchuk, Y. V., Shelest, T. N. & Smirnoff, S. V. Brain Res. 436: 352-356 (1987), with sustained components. Both ASIC1a, Waldmann, R., Champigny, G., Bassilana, F., Heurteaux, C. & Lazdunski, M. Nature 386: 173-177 (1997), and ASIC2a, Champigny, G., Voilley, R., Waldmann, R. & Lazdunski, M. J. Biol. Chem. 273: 15418-15422 (1998), desensitize within a few seconds during prolonged application of extracellular acid, Waldmann, R., Champigny, G., Lingueglia, E., De Weille, J. R., Heurteaux, C. & Lazdunski, M. Annals New York Academy Of Sciences 67-76 (Apr. 30, 1999). Pain associated with tissue acidosis, however, continues until the pH returns to neutral, Steen, K. H., Issberner, U. & Reeh, P. W. Neurosci. Lett 199: 29-32 (1995), Waldmann, R., Champigny, G., Lingueglia, E., De Weille, J. R., Heurteaux, C. & Lazdunski, M. Annals New York Academy Of Sciences 67-76 (Apr. 30, 1999). A biphasic H⁺-gated cation current with a sustained component was described in sensory neurons, Bevan, S. & Yeats, J. J. Physiol. 433:145-161 (1991), and was proposed to be responsible for the nonadapting pain with tissue acidosis, Bevan, S. & Yeats, J. J. Physiol. 433:145-161 (1991), Reeh, P. W. & Steen, K. H. Prog. Brain. Res. 113: 143-151 (1996), Waldmann, R., Champigny, G., Lingueglia, E., De Weille, J. R., Heurteaux, C. & Lazdunski, M. Annals New York Academy Of Sciences 67-76 (Apr. 30, 1999). The specific expression of ASIC3 in sensory neurons and the kinetics of the ASIC3 channel suggest that it is part of the sustained H⁺-gated cation channel complex in sensory neurons. The sustained ASIC3 current, however, requires a more acidic pH for activation (<pH 4) than the native H+-gated current in sensory neurons, Bevan, S. & Yeats, J. J. Physiol. 433:145-161 (1991), (pH=5.8). This suggests that a translational modification or associated subunits are required to form the native H⁺-gated cation channel. WO 00/08149 discloses the cloning of the rat and human ASIC3 proteins.

Another ASIC channel is ASIC4. ASIC4 is a new protein showing about 45% identity to other ASICs. ASIC4 is 97% identical between rat and human and shows strongest expression in pituitary gland, Stefan Gruender, Hyun-Soon Geissler, Eva-Lotta Baessler, Peter Ruppersberg, NeuroReport, Vo.11, No. 85: 1607-16211 (June, 2000). A drop of extracellular pH in Xenopus oocytes cannot activate ASIC4, suggesting association with other subunits or activation by a ligand different from protons.

In brief, ASIC2b (MDEG2) is present in sensory neurons where it modulates the expression of ASIC3 (DRASIC). Coexpression of the two proteins yields a H⁺-gated current that contains a non-selective sustained component. Thus, it seems very probable that these two units, ASIC2b and ASIC3, constitute at least part of the native proton-gated cation channel of nociceptive neurons, Bevan, S. & Yeats, J. J. Physiol. 433:145-161 (1991), Lingueglia, E., De Weille, J. R., Bassilana, F., Heurteaux, C., Sakai, H., Waldmann, R. & Lazdunski, M. J. Biol. Chem. 272: 29778-29783 (1997), Waldmann, R., Champigny, G., Lingueglia, E., De Weille, J. R., Heurteaux, C. & Lazdunski, M. Annals New York Academy Of Sciences 67-76 (Apr. 30, 1999).

Thus, as the modulation of ASICs can have therapeutic consequences for the human, there is a continued need to provide a new ASIC and its agonists and antagonists. In particular, since hASIC2b and the other hASICs constitute at least part of the native proton-gated cation channel of nociceptive neurons, it is necessary to provide a novel method for screening a candidate substance that can modulate the ASICs by bringing it into contact with transformed cells in which both hASIC2b and the other hASICs have been coexpressed.

### SUMMARY OF THE INVENTION

In a broad aspect, the present invention relates to novel nucleic acid sequences encoding human ASIC2b. In this regard, a specific novel nucleic acid sequence has been isolated and it is to be understood that the invention covers that sequence as well as novel variants, fragments, derivatives, and homologues thereof.

In another aspect, the present invention relates to novel amino acid sequences. In this regard, a specific novel amino acid sequence has been isolated and it is to be understood that the invention covers that sequence as well as novel variants, fragments, derivatives, and homologues thereof.

Thus, in brief, some aspects of the present invention relate to:

1. Novel nucleotide sequences;

2. Novel amino acids;

3. Assays using said novel sequences;

4. Compounds/compositions identified by use of said assays;

5. Expression systems comprising or expressing said novel sequences optionally together with the other ASICs;

6. Methods of treatment based on said novel sequences;

7. Pharmaceutical compositions based on said novel sequences.

Other aspects concerning the nucleotide sequence of the present invention and/or the amino acid sequence of the present invention include: a construct comprising or capable of expressing the sequences of the present invention; a vector comprising or capable of expressing the sequences of the present invention; a plasmid comprising or capable of expressing the sequences of the present invention; a cell transfected or virally-transduced with a construct/vector/plasmid comprising or capable of expressing the sequences of the present invention; a tissue comprising or capable of expressing the sequences of the present invention; an organ comprising or capable of expressing the sequences of the present invention; a transformed host comprising or capable of expressing the sequences of the present invention; and a transformed organism comprising or capable of expressing the sequences of the present invention. The present invention also encompasses methods of expressing the same, such as expression in a microorganism; including methods for transferring the same.

### BRIEF DESCRIPTION OF THE FIGURES

FIG. 1A and FIG. 1B show the alignment of deduced protein sequences of hASIC2a (at top) and hASIC2b (at bottom);

FIG. 2A, FIG. 2B, FIG. 2C, FIG. 2D, FIG. 2E, FIG. 2F, FIG. 2G, and FIG. 2H show the nucleotide sequence of hASIC2b;

FIG. 3A, FIG. 3B, FIG. 3C, and FIG. 3D show the amino acid sequence of hASIC2b;

FIG. 4 shows the tissue distribution of human ASIC2b and ASIC2a;

FIG. 5 shows the whole-cell recording from hASIC2b, hASIC2a, and hASIC2a/hASIC2b expressing CHO-K1 cells; and

FIG. 6A shows the electrophysiological properties of acid-sensing current in hASIC2a/hASIC2b co-expressing CHO-K1 cells; The pH dependence of acid-sensing currents in hASIC2a and hASIC2a/hASIC2b co-expression CHO-K1 cells; and FIG. 6B shows comparison of peak current density in hASIC2a and hASIC2a/hASIC2b CHO-K1 transformants.

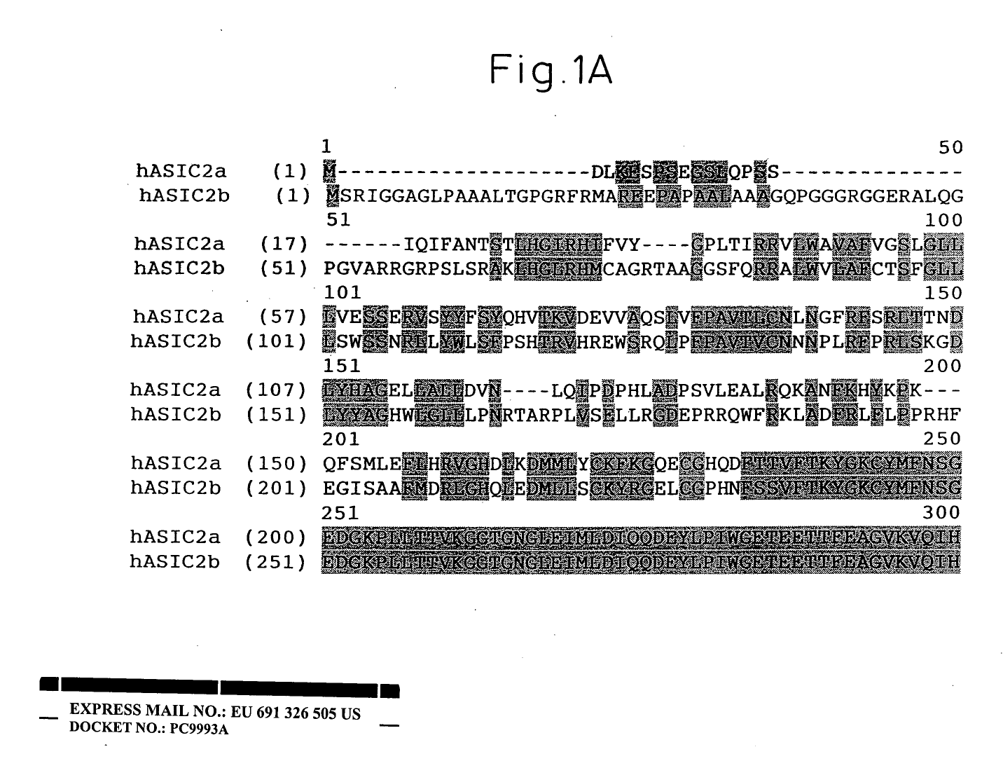

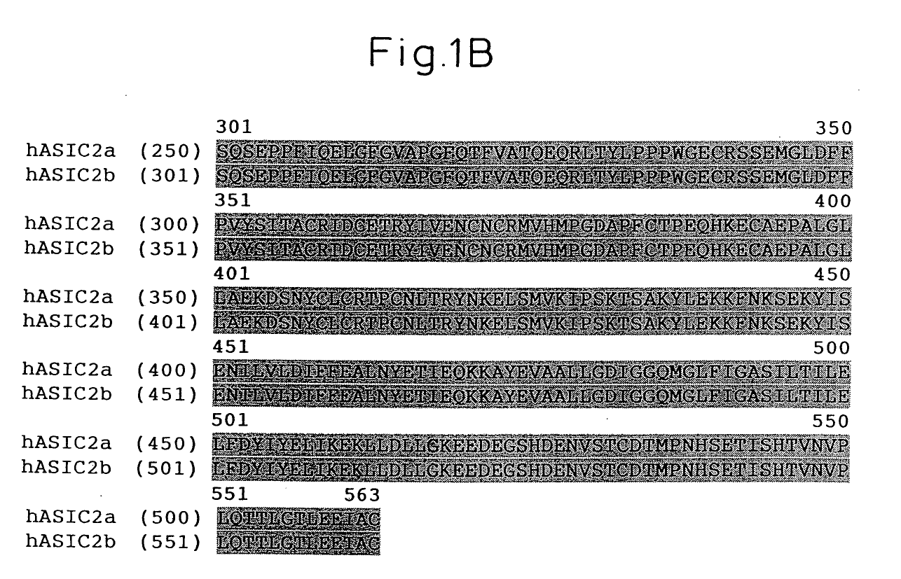

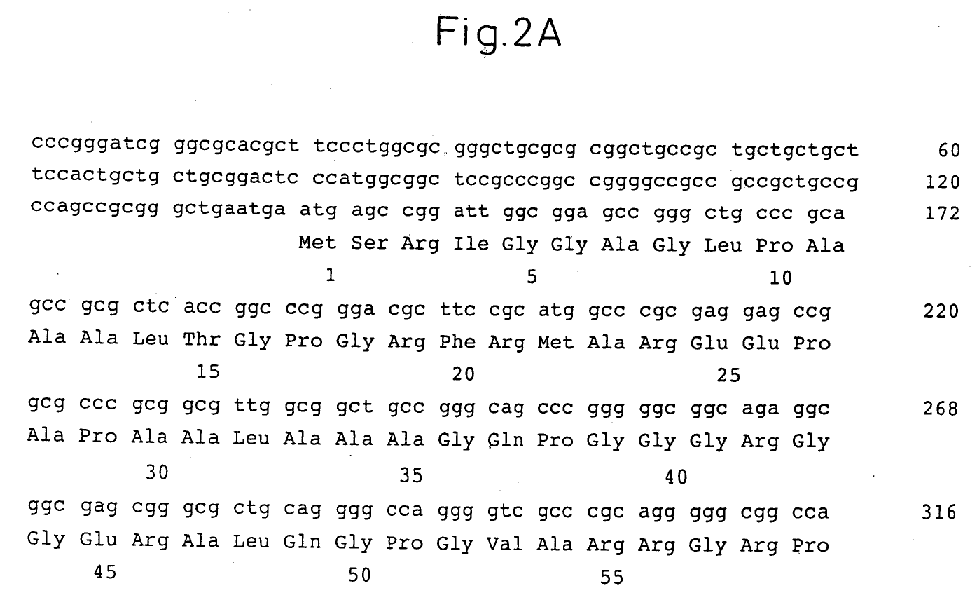

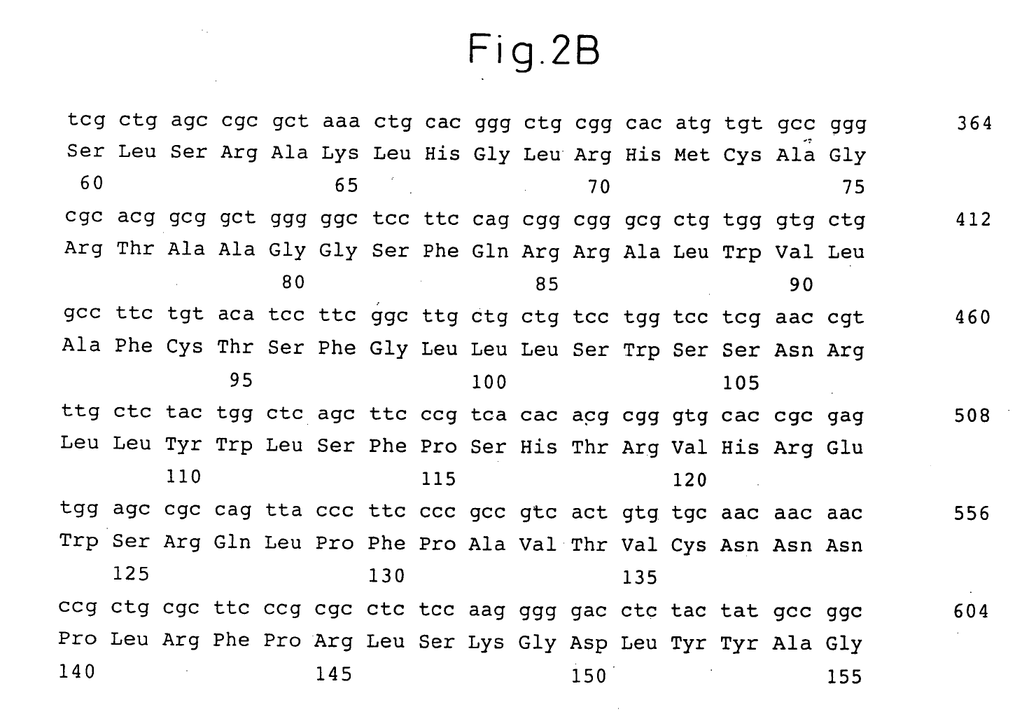

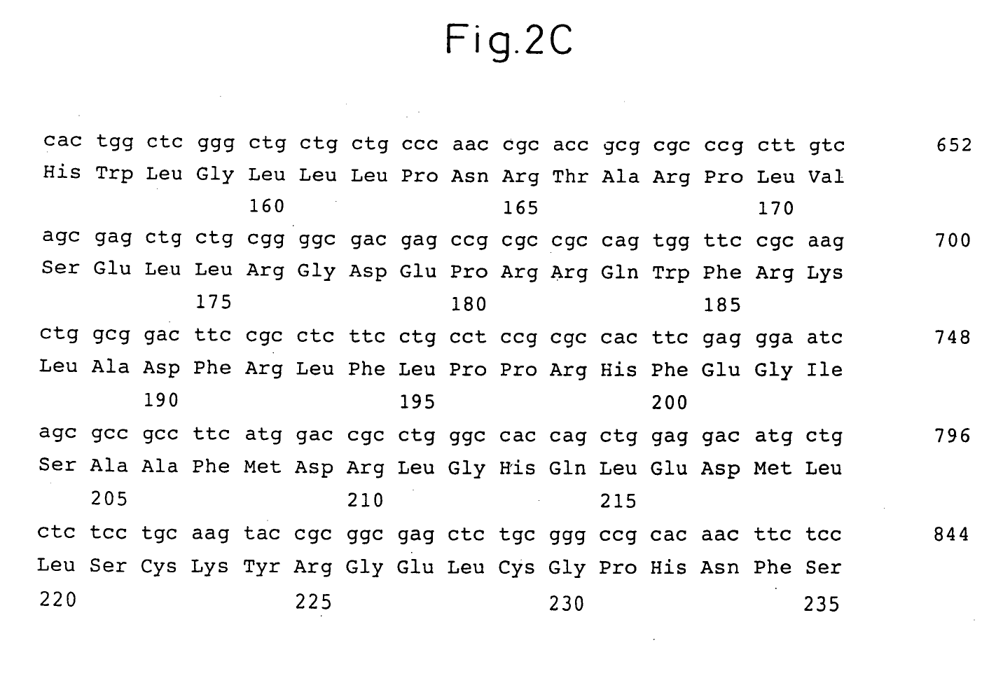

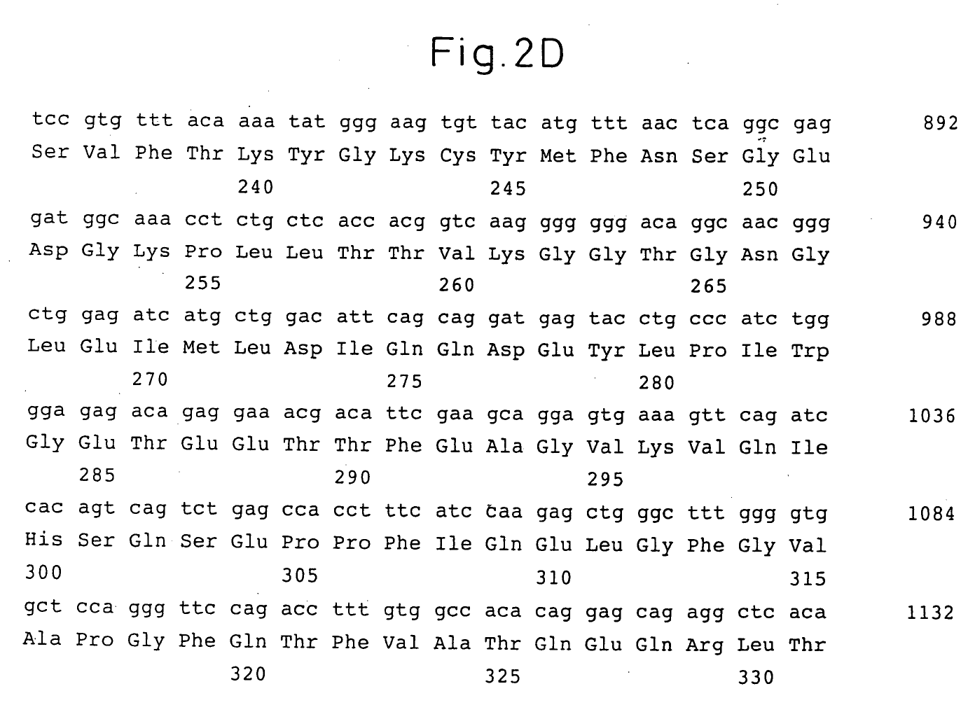

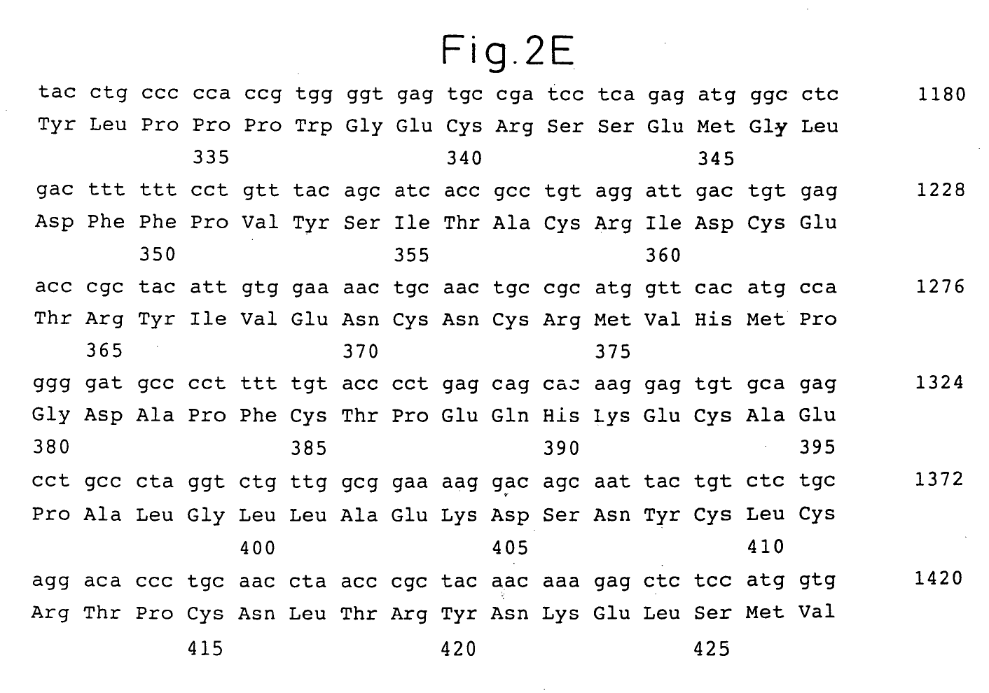

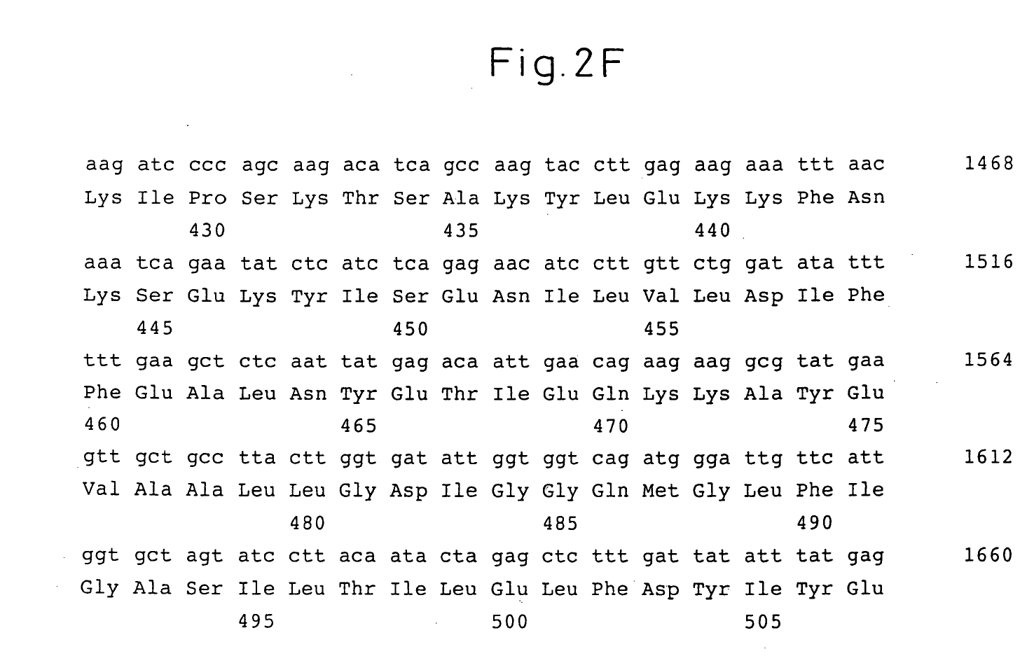

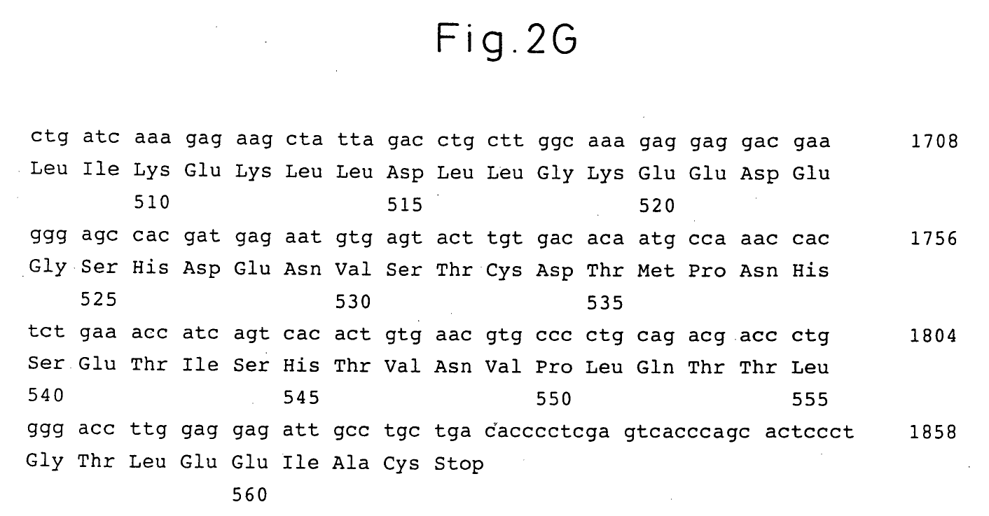

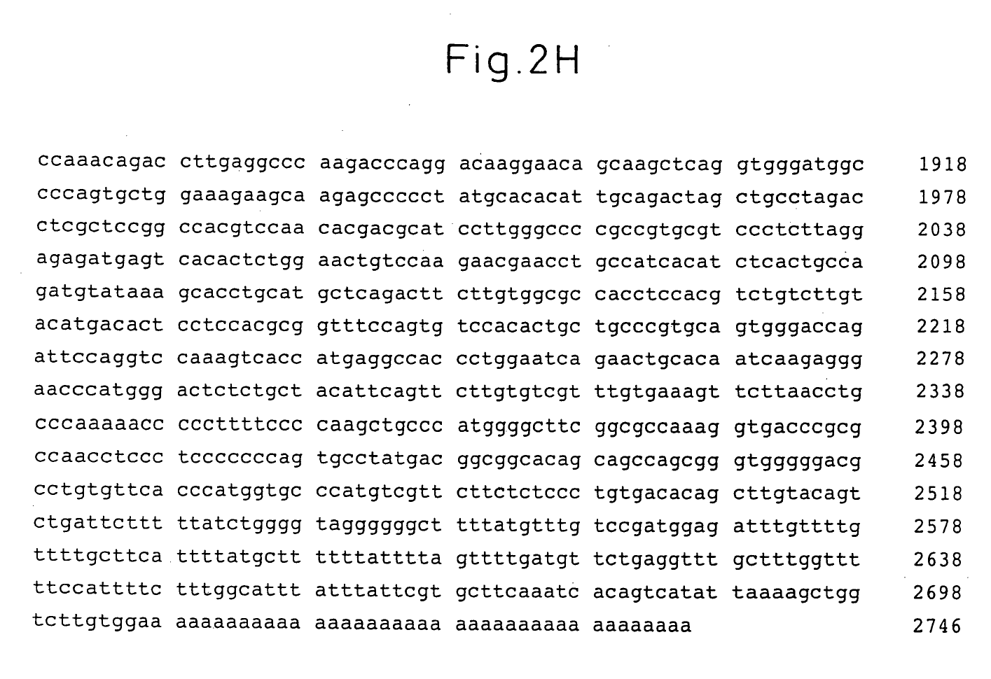

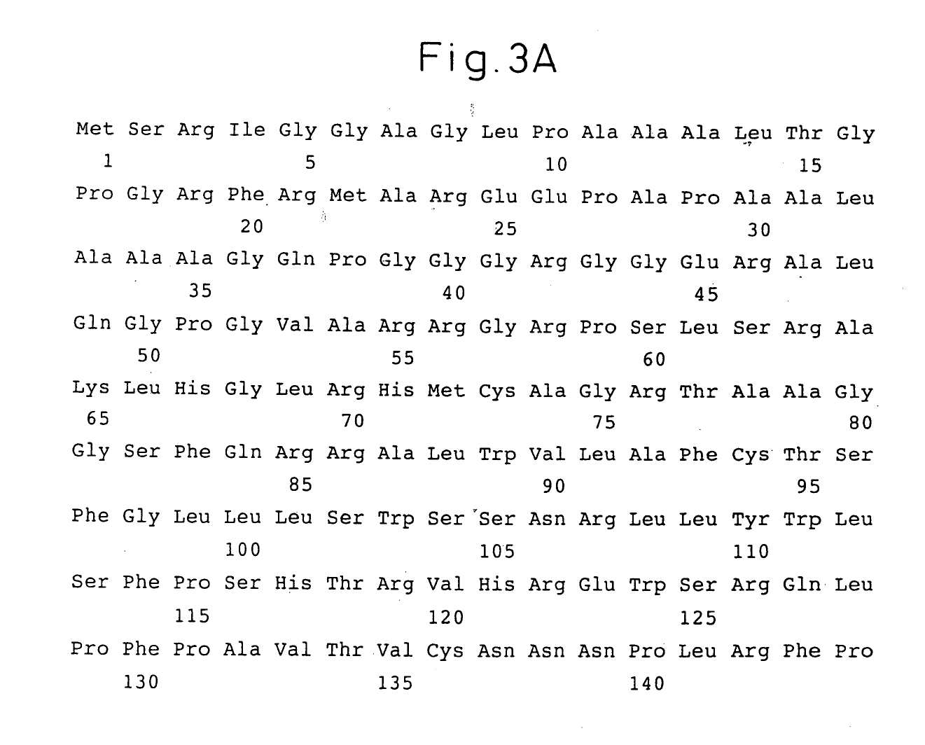

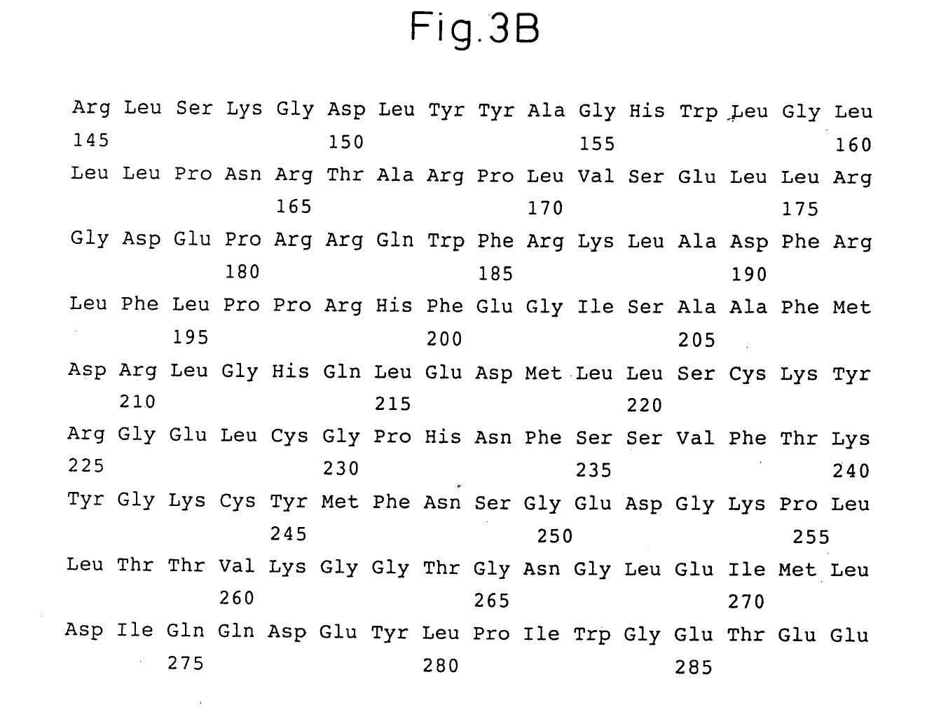

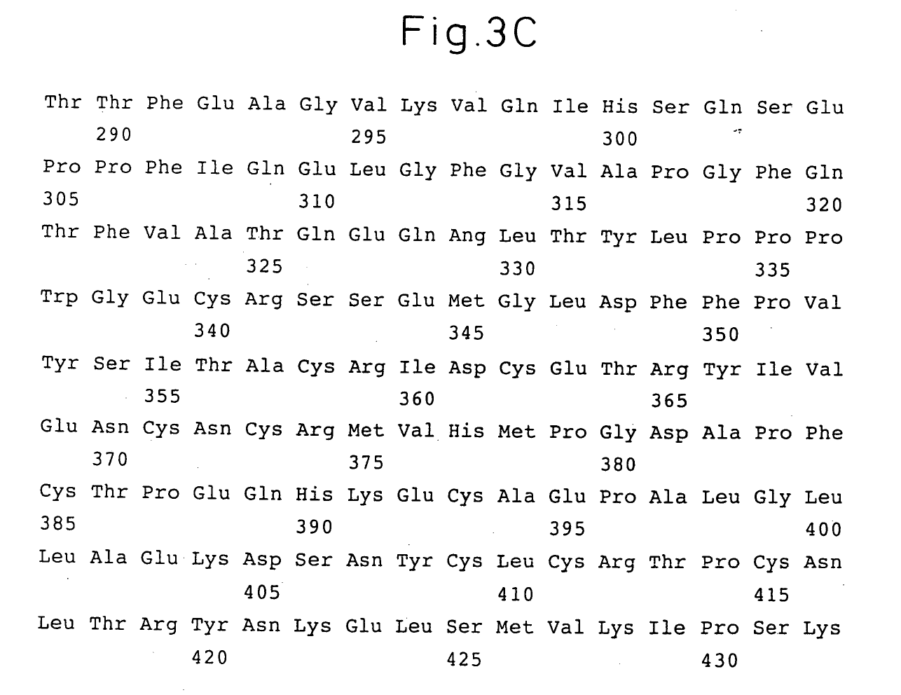

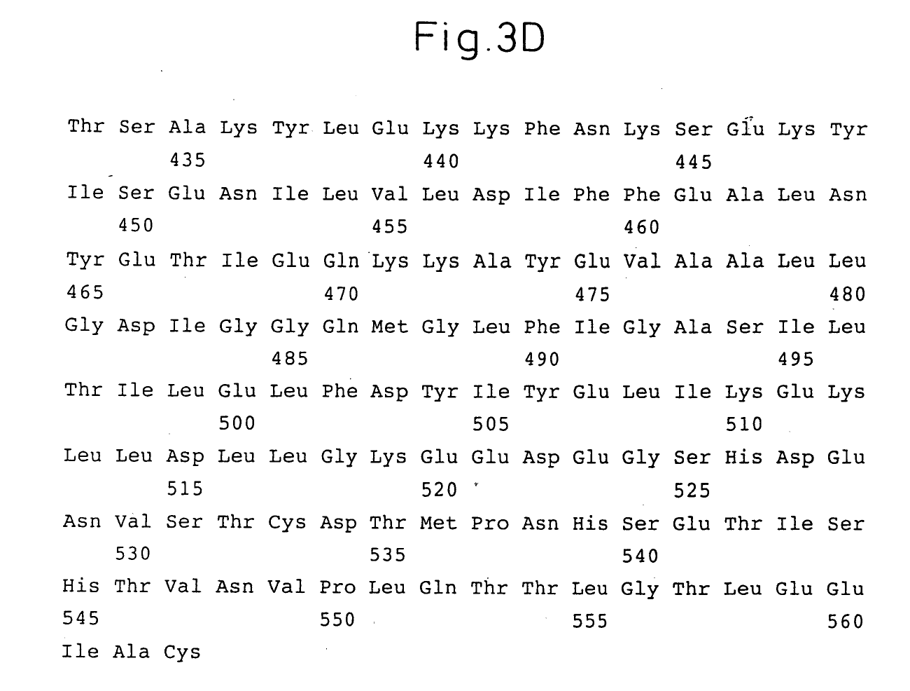

### IDENTIFICATION OF THE SEQUENCE LISTINGS

SEQ ID NO: 1 shows the nucleotide sequence coding for hASIC2b;

SEQ ID NO: 2 shows the corresponding amino acid sequence coding for hASIC2b;

SEQ ID NO: 3 shows oligonucleotide probe used in the GENE GENE TRAPPER III experiments;

SEQ ID NO: 4 shows oligonucleotide probe used in the GENE GENE TRAPPER III experiments;

SEQ ID NO: 5 shows oligonucleotide probe used in the GENE GENE TRAPPER III experiments;

SEQ ID NO: 6 shows the sense primer for hASIC2b;

SEQ ID NO: 7 shows the antisense primer for hASIC2b;

SEQ ID NO: 8 shows the sense primer for hASIC2a;

SEQ ID NO: 9 shows the antisense primer for hASIC2a;

SEQ ID NO: 10 shows the sense primer for GAPDH; and

SEQ ID NO: 11 shows the antisense primer for GAPDH.

### DETAILED DESCRIPTION OF THE INVENTION

According to one aspect of the present invention, there is provided a polynucleotide comprising one or more of:

(a) a polynucleotide encoding the polypeptide as set forth in SEQ ID NO:2;

(b) a polynucleotide comprising a nucleotide sequence of SEQ ID NO:1;

(c) a polynucleotide comprising a nucleotide sequence that has at least 70% identity to the polynucleotide of (a) or (b);

(d) a polynucleotide comprising a nucleotide sequence which is capable of hybridizing to the polynucleotide of any one of (a) to (c);

(e) a complement to the polynucleotide of any one of (a) to (d); or

(f) a polynucleotide fragment of the polynucleotide of any one of (a) to (e).

Preferably the polynucleotide is isolated and/or purified. Preferably, the polynucleotide comprises a nucleotide sequence that has at least 75% identity to the polynucleotide of (a) or (b). More preferably, the polynucleotide comprises a nucleotide sequence that has at least 80% identity to the polynucleotide of (a) or (b). Even more preferably, the polynucleotide comprises a nucleotide sequence that has at least 85% identity to the polynucleotide of (a) or (b). Yet more preferably, the polynucleotide comprises a nucleotide sequence that has at least 90% identity to the polynucleotide of (a) or (b). More preferably, the polynucleotide comprises a nucleotide sequence that has at least 95% identity to the polynucleotide of (a) or (b).

The polynucleotide described above preferably encodes a human acid sensing ion channel (ASIC) 2b.

The present invention yet further provides a vector comprising the polynucleotide described above.

According to a further aspect of the present invention, there is provided a host cell transformed or transfected with the vector described above. Preferably, the host cell is mammalian, insect, fungal, bacterial or yeast cell.

According to a further aspect of the present invention, there is provided the transcribed RNA product of the polynucleotide described above. There is also provided an RNA molecule or fragment thereof, which is antisense in relation to the RNA product and is capable of hybridizing to the RNA product.

There is yet further provided a ribozyme or zinc finger protein capable of binding the polynucleotide described above.

According to a yet further aspect of the present invention, there is provided a process of producing a polypeptide or fragment thereof comprising culturing the transformed/transfected host cell under conditions sufficient for the expression of said polypeptide or fragment. Preferably, said polypeptide or fragment is expressed at the surface of said cell. The process preferably further includes recovering the polypeptide of fragment from the culture.

There is also provided by the present invention a process of producing cells capable of expressing a polypeptide or fragment thereof comprising transforming or transfecting cells with the vector described above.

According to a further embodiment of the present invention, there are provided cells produced by the process described above. There is also provided a membrane preparation of said cells.

According to another aspect of the present invention, there is provided a polypeptide or a fragment thereof produced by the process described above.

According to another aspect of the present invention, there is provided a polypeptide comprising:

(a) a polypeptide having the deduced amino acid sequence translated from the polynucleotide sequence in SEQ ID NO:1 and variants, fragments, homologues, analogues and derivatives thereof; or

(b) a polypeptide of SEQ ID NO:2 and variants, fragments, homologues, analogues and derivatives thereof.

There is also provided by the present invention said polypeptide fused with another human acid sensing ion channels (hASICs). Preferably, said another hASICs may be selected from the group consisting of hASIC1a, hASIC1b, hASIC2a, hASIC3, hAISC4, and their derivatives.

There is also provided an antibody against the polypeptides described above.

The present invention yet further provides a compound, which modulates the polypeptide described above. Preferably, the compound antagonizes or selectively antagonized the polypeptide. Alternatively, the compound agonizes the polypeptide.

According to another aspect of the present invention, there is provided a method of screening for substances capable of modulating the polypeptide described above, which comprises:

(a) contacting a substance to be tested with cells expressing at least one molecule of said polypeptide and optionally at least one molecule of an additional human acid sensing ion channel (hASIC) selected from the group consisting of hASIC1a, hASIC1b, hASIC2a, hASIC3, hAISC4, and their derivatives on their surface;

(b) measuring the effects of the substance on the transport functions of said polypeptide and/or at least one of said hASICs and derivatives; and

(c) identifying the substances that have a positive or negative effect on the transport functions.

Preferably the substance to be tested is in a preselected amount.

According to another aspect of the present invention, there is provided a method of identifying a compound, which binds to and modulates the polypeptide described above, comprising contacting said polypeptide with a candidate compound and determining whether modulation occurs.

Preferably, said method comprises:

(a) contacting a compound with cells expressing at least one molecule of the polypeptide described above and optionally at least one molecule of an additional human acid sensing ion channel (hASIC) selected from the group consisting of hASIC1a, hASIC1b, hASIC2a, hASIC3, hAISC4, and their derivatives on their surface, said polypeptide or at least one of said hASICs or derivatives being associated with a second component capable of providing a detectable signal in response to the binding of a compound to said polypeptide or at least one of said hASICs or derivatives; said contacting being under conditions sufficient to permit binding of compounds to the polypeptide or at least one of said hASICs or derivatives; and

(b) identifying a compound capable of binding the polypeptide or at least one of said hASICs or derivatives by detecting the signal produced by said second component.

Preferably the compound binds to and (i) antagonizes or selectively antagonizes the polypeptide described above, or (ii) agonizes the polypeptide of described above.

As hASICs are involved in cation transport, modulators (e.g. agonists or antagonists) of the polypeptide of the present invention can find use in interfering with the cation transport process.

Therefore, according to yet another embodiment of the present invention, there is provided the antibody or compound described above for use as a pharmaceutical.

Such antibodies, and compounds, etc., which can modulate the polypeptide of the present invention, can therefore find use in the therapeutic areas which concern aspects of cation transport. Therapeutically useful areas include, but are not limited to, disorders of perception of acidity with regard to nociception and taste transduction, pain, disorders of acid taste, neurodegeneration induced by hyperexpression of ASICs, cerebral neuronal degeneration, Alzheimer's disease, Huntington's disease, Parkinson's disease, amyotrophic lateral sclerosis, cerebellar ataxia, inflammatory diseases, ischemia, and certain tumors.

Accordingly, there is also provided the use of the compound described above in the manufacture of a medicament for the treatment of a patient having need to modulate the polypeptide described above. Preferably, the treatment is for a patient having a need to antagonize or selectively antagonize the polypeptide. Alternatively, the treatment is for the treatment of a patient having a need to agonize the polypeptide.

According to a yet further aspect of the invention, there is provided a method for the treatment of a patient having need to modulate the polypeptide comprising administering to the patient a therapeutically effective amount of the compound. Preferably, said method is for the treatment of a patient having a need to antagonize or selectively antagonize the polypeptide. Alternatively, said method is for the treatment of a patient having a need to agonize the polypeptide.

Preferably, said compound is a polypeptide and a therapeutically effective amount of the compound is administered by providing to the patient DNA encoding said compound and expressing said compound in vivo.

There is also provided, by the present invention, use of the antibody described above in the manufacture of a medicament for the treatment of a patient having a need to modulate the polypeptide described above. Preferably, said method is for the treatment of a patient having a need to antagonize or selectively antagonize the polypeptide. Alternatively, said method is for the treatment of a patient having a need to agonize the polypeptide.

Yet further provided by the present invention is a method for the treatment of a patient having a need to modulate the polypeptide described above, comprising administering to the patient a therapeutically effective amount of the antibody described above. Preferably, said method is for the treatment of a patient having a need to antagonize or selectively antagonize the polypeptide. Alternatively, said method is for the treatment of a patient having a need to agonize the polypeptide.

According to a yet further aspect of the present invention, there are provided cells genetically engineered ex vivo or in vivo to express, overexpress, underexpress or to exhibit targeted insertion or deletion of the polypeptide of the present invention. There is also provided by the present invention a transgenic non-human animal comprising such cells.

As discussed above, ASIC2b is considered a modulator subunit of acid sensing ion channels in brain and DRGs, Lingueglia, E., De Weille, J. R., Bassilana, F., Heurteaux, C., Sakai, H., Waldmann, R. & Lazdunski, M. J. Biol. Chem. 272: 29778-29783 (1997). RASIC2b is not active by itself, but it can associate with either rASIC2a or rASIC3 to modify their properties. For example, it confers non-selectivity to late H⁺-induced current. RASIC2b is considered to interact with rASIC2a to form heteromultimers with new properties, Lingueglia, E., De Weille, J. R., Bassilana, F., Heurteaux, C., Sakai, H., Waldmann, R. & Lazdunski, M. J. Biol. Chem. 272: 29778-29783 (1997). It has also been shown that the rASIC3 current, like the native proton-gated current in dorsal root sensory neurons, consists of two components: a rapid inactivation current followed by a sustained current, Waldmann, R., Bassilana, F., De Weille, J., Champigny, G., Heurteaux, C. & Lazdunski, M. J. Biol. Chem. 272: 20975-20978 (1997). It has also been shown that coexpression of rASIC2b and rASIC3 yields a current that looks like a rASIC3-like current, Waldmann, R., Bassilana, F., De Weille, J., Champigny, G., Heurteaux, C. & Lazdunski, M. J. Biol. Chem. 272: 20975-20978 (1997). RASIC2b is present in sensory neurons where it modulates the expression of rASIC3. Coexpression of the two proteins yields a H⁺-gated current that contains a non-selective sustained component. Thus, it is very probable that these two units, rASIC2b and rASIC3 are at least part of the native proton-gated cation channel of nociceptive neurons, Bevan, S. & Yeats, J. J. Physiol. 433:145-161 (1991), Lingueglia, E., De Weille, J. R., Bassilana, F., Heurteaux, C., Sakai, H., Waldmann, R. & Lazdunski, M. J. Biol. Chem. 272: 29778-29783 (1997), Waldmann, R., Champigny, G., Lingueglia, E., De Weille, J. R., Heurteaux, C. & Lazdunski, M. Annals New York Academy Of Sciences 67-76 (Apr. 30, 1999).

The amino acid sequence homologies of human ASICs are shown in Table 1 below.

TABLE 1
Amino acid sequence homologies of human ASICs

| | ASIC3 | ASIC1 | ASIC2a | ASIC2b | ASCI4 |
|---|---|---|---|---|---|
| ASIC3 | 100 | 48.0 | 48.7 | 45.6 | 48.6 |
| ASIC1 | | 100 | 72.0 | 60.3 | 49.7 |
| ASIC2a | | | 100 | 80.6 | 50.4 |
| ASIC2b | | | | 100 | 48.1 |
| ASIC4 | | | | | 100 |

Large changes in extracellular acidity are produced in the brain in the course of ischemia and epileptic seizures. Therefore, this class of ASIC-type channels will certainly be activated in these pathophysiological conditions. This activation would be expected to produce deleterious effects. The effects include cellular depolarization and a significant contribution to the well-known massive Na⁺entry, which occurs, especially in ischemia, when the (Na⁺, K⁺) ATPase will be less active in pumping Na⁺out because of intracellular ATP depletion. Blockers of the H⁺-gated cation channels that are more specific than amiloride would be important in studying the role of those channels both in pain perception and in physiological and pathophysiological brain functions. Such specific inhibitors have not yet been available, but the search for such blockers will be greatly facilitated with the availability of cDNA clones including hASIC2b cDNA clones of the present invention.

The polypeptide of hASIC2b can be useful for developing a medicament for the treatment or prevention of pathologies entailing the painful perception of acidity found in inflammatory diseases, ischemia, and certain of tumors.

The present invention also provides the transformed cells expressing hASIC2b of the present invention and optionally at least one of other hASICs or their derivatives. These cells are useful for screening candidate substances that are capable of modulating cation transport by these polypeptides and therefore the perception of acidity with regard to both nociception and taste transduction. This screening can be carried out by bringing a predetermined amount of a substance to be tested into contact with the cells (co)-expressing the hASIC channels and determining the effects of said substance on the currents of said cation channels. These screenings allow for the identification of new drugs that are useful in the treatment or prevention of pain such as analgesics. They also enable the identification of agents that modulate acid taste.

The substances that are isolated and detected by means of the methods described above are also part of the present invention. Thus, the present invention also provides a chemical or biological substance that is capable of modifying the currents of an ionic channel and/or a hybrid channel according to the present invention in the manufacture of a medicament capable of modulating the perception of acidity with regard to nociception as well as taste transduction in a human or animal subject.

The polynucleotide coding for hASIC2b of the present invention or derivative thereof, or a vector comprising the polynucleotide or a cell expressing hASIC2b is also useful for the preparation of non-human transgenic animals used in developing a new drug. These transgenic non-human animals can be those overexpressing or underexpressing said channels, but also “knock-out” animals either deficient in the expression of these channels or in the cation transport activity of these channels. These non-human transgenic animals are prepared by the methods, per se, known in the art, and serve as live animal models in studying pathologies associated with ASIC channels.

The polynucleotide of the present invention or the cells transformed with said polynucleotide can also be used for genetic therapy to compensate for a deficiency in hASIC2b channel at a certain tissue of a patient. Thus, the present invention also provides a drug comprising the polynucleotide of the present invention or the cells transformed by said polynucleotide for the treatment of pathology involving hASIC2b or its derivatives.

In addition to the property of being activated by protons and the resultant applications described above relating to the perception of acidity, hASIC2b having genetic mutations may be involved in some neurodegenerative processes. The death of certain neurons is characteristic of many types of neuronal degenerative disorders such as Alzheimer's disease, Huntington's disease, Parkinson's disease, amyotrophic lateral sclerosis, and cerebellar ataxia. Only a few deficient genes involved in such neurodegenative processes have been identified. The primitive neural network of the nematode C. elegans is a good model of neuronal development and death. The hereditary degeneration in C. elegans can be due to mutations of the genes deg-1, mec-4, and mec-10. ASIC2a is activated by the same mutations, Waldmann, R., Champigny, G., Voilley, N., Lauritzen, I. & Lazdunski, M. J. Biol. Chem. 271, 10433-10436 (1996).

Therefore, the present invention provides a use of hASIC2b channel in studying these pathological modifications that may lead to neuronal degenerations. The screening methods discussed above are useful for identifying substances that can block or inhibit neurodegeneration induced by overexpression or undrexpression of these channels. The ASIC channels have ionic properties in terms of selective permeability by sodium, potassium, lithium, and calcium. The selective permeability may cause excitotoxicity when said ASIC channels are hyperstimulated.

The polypeptide of hASIC2b, an agonist or antagonist of said protein can also be used in the manufacture of a medicament for the treatment of prevention of pathologies involving cerebral neuronal degenerations.

Other characteristics and advantages of the present invention will be seen in the Examples below related to research activities that led to the demonstration and the characterization of hASIC2b channel of the present invention, and in which reference will be made to the annexed sequences and figures.

### EXAMPLES

### Example 1

### Cloning of Human ASIC2b cDNA

Total RNA samples isolated from human dorsal root ganglia (hDRG) was purchased from Analytical Biological Services Inc. (Wilmington, Del.), and hDRG cDNA library that contains a total of 1.5×10⁷ clones of a size-fractionated (average length: 2.0 kb) oligo (dT)-primed was constructed in pCMVSPORT6 by Life Technologies Inc.

GENE TRAPPER III cDNA Positive Selection System (Life Technologies Inc.) was used to screen novel ASIC clones. Experiments were performed according to the manufacturer's instructions. Degenerate oligonucleotide probes were designed by the alignment of four published human acid sensing ion channel (ASIC) polypeptide sequences (GenBank accession numbers: AF095897, AF057711, AB010575, and NM_{—}001094). Three oligonucleotide probes (A1: 5′-TTY CCR GCN GTN ACC CCT STG YA-3′ (SEQ ID NO: 3); A4: 5′-CTG GAC RTK CAN CAN GAN GAR T-3′ (SEQ ID NO: 4); and A9: 5′-GGN YTK TTY ATH GGK GCY AG-3′ (SEQ ID NO: 5)) were selected and used in the GENE TRAPPER III experiments and colony hybridization. The degenerate probes were biotinylated by TdT and Biotin-14-dCTP (Life Technlodies Inc.) at 30° C. for 1 hr., and the biotinylation of oligonucleotide probes were confirmed by 15% TBE/Urea polyacrylamide gel electrophoresis (Novex). The single-stranded cDNA (ssDNA) was generated from the double-stranded hDRG cDNA library clones with Gene II and Exonuclease III (Life Technologies Inc.) at 30° C. for 30 min. The biotinylated ologonucleotide and ssDNA were hybridized at 37° C. for 1 hr. Streptavidin paramagnetic beads were added to the hybridization mixture to capture the ssDNA hybridized to the biotinylated probes at room temperature for 30 min. The captured ssDNA were repaired using TP-3000 thermal cycler (TaKaRa) and the Repair Enzymes (Life Technologies Inc.). Repair reaction was carried out with the thermal cycler for one cycle (denaturing step at 90° C. for 1 min., annealing step at 55° C. for 30 seconds, extension step at 70° C. for 15 min. and soaking step at 4° C.). E. coli. strain DH5α(Life Technologies Inc.) was transformed with repaired cDNAs, and tranferred onto Hybond-N (Amersham) filters prior to hybridization. The cDNA on filters were denatured in the denaturing solution (0.5N NaOH and 1.5M NaCl) at room temperature for 7 min. and neutralized twice in the neutralizing solution (1.5M NaCl and 0.5M; Tris-HCl, pH adjusted to 7.5) at room temperature for 3 min. The filters were washed with 2×SSC at room temperature for 2 min. The denatured cDNAs on the filters were immobilized by using the CL-1000 ultraviolet cross linker (UVP).

The degenerate oligonucleotide probes were labeled at the 3′-end with fluorescein-dUTP using the Gene Images 3′-oligolabelling kit (Amersham) and hybidization was carried out in the ExpressHyb Hybridization Solution (CLONTECH) at 42° C. for 1 hr. The filters were washed twice in 5×SSC with 0.1% SDS at room temperature for 5 min., then in 1×SSC with 0.1% SDS at 42° C. for 15 min. Positive clones were selected using the Gene Images CDP-Star detection kit (Amersham) and LAS-1000 imaging system (Fuji Film) according to the manufacturer's instructions. Positive clones were picked up, and their nucleotide sequences were determined in the CEQ2000 DNA analyzer (Beckman). The sequences were analyzed by BLAST search. Among the clones belonging to the ASIC family, a novel splice variant of human (h)ASIC2 with a unique N-terminal 236 amino acids (aa) was discovered, which contained an open reading frame of 1,689 base pairs encoding a protein of 563 aa. This clone was designated as human ASIC2b. The nucleotide sequence and amino acid sequence of hASIC2b are shown in SEQ ID NO: 1.

### Example 2

### Expression Profiling Human ASIC2b AND ASIC2a

Material and Methods

Expression of human ASIC2a and human ASIC2b transcripts were examined by Reverse Transcription-polymerase chain reaction. Total RNA samples from various human tissues (CLONTECH and ABS) were used in the reverse transcription reaction. An aliquot of 2 μg of total RNA was primed with oligo(dT)₁₂₋₁₈ and reverse-transcribed with SuperScript II (Life Technologies Inc.) in a total volume of 20 μl. Polymerase chain reaction was performed with 0.5 μl of the first strand cDNA in a reaction volume of 20 μl.

Primers used were (5′-3′, sense/antisense)

| | | |
|---|---|---|
| hASIC2b: | CTG CTC TCC TGC AAG TAC C/ | (SEQ ID |
| | | NO: 6) |
| | AGC TCT TGG ATG AAA GGT GGC; | (SEQ ID |
| | | NO: 7) |
| and | | |
| hASIC2a: | ACC ACC AAC GAC CTG TAC C/ | (SEQ ID |
| | | NO: 8) |
| | AGA GGT TTG CCA TCC TCG C. | (SEQ ID |
| | | NO: 9) |

PCR was performed under the following conditions: PCR conditions were: hASIC2b (94° C. for 1 min; 35 cycles of 94° C. for 20 seconds, 56° C. for 20 seconds, 72° C. for 20 seconds; 72° C. for 5 min), and hASIC2a (94° C. for 1 min; 30 cycles of 94° C. for 20 seconds, 60° C. for 20 seconds, 72° C. for 20 seconds; 72° C. for 5 min). PCR amplification of glyceraldehyde-3-phosphate dehydrogenase (GAPDH) mRNA was also performed as a control experiment. The sequences of GAPDH-specific primers are as follows:

(5′-3′, sense/antisense) GTC TTC ACC ACC ATG GAG
AAG GCT (SEQ ID NO: 10)/GTG ATG GCA TGG ACT GTG GTC
ATG A (SEQ ID NO: 11).

One-half of the PCR products were electrophoresed on a 2% TAE-agarose gel, stained with ethidium bromide, and photographed under UV light.

Results

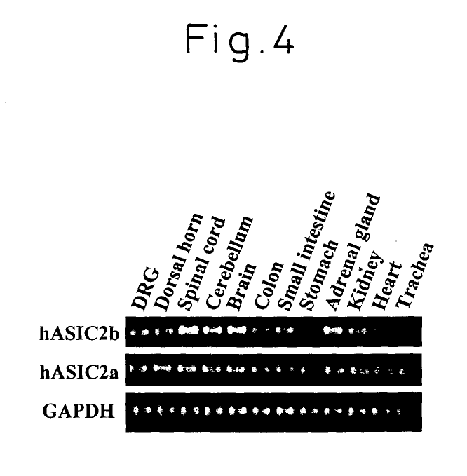

As can be seen from FIG. 4, transcripts of hASIC2a and hASIC2b were detected in most human tissues examined. Expression of hASIC2a is equally distributed in all tissues examined, however, expression of hAISC2b is highly expressed in neuronal tissues such as spinal cord, brain, and DRG, and adrenal gland and small intestine. These results suggest an important role of hASIC2b in neuronal functions.

### Example 3

### Functional Anaylsis of hASIC2b

Materials and Methods

The mammalian expression vectors for hASIC2b and hASIC2a were constructed using appropriate expression vectors such as pcDNA3.1 (Clonetech) according to conventional molecular biological methods. Chinese Hamster Ovary (CHO)-K1 cells were seeded on a 35 mm dish in diameter at a density of 20,000 cells, and then transfected with various combinations of ASIC expression vectors with FuGENE6 transfection reagent (Roche) according to the manufacturer's instructions as follows: the hAISC2b expression vector alone (1 μg) for homomeric hASIC2b expression, hASIC2a and green fluorescent protein (GFP) expression vectors (1:2 molar ratio in a total of 1 μg) for hASIC2a expression, and hASIC2b/hASIC2a (1:2 molar ratio in a total of 1 μg) for heteromeric expression. Cells were used for electrophysiological measurements 2 days after the transfection. Successfully transfected cells were recognized by GFP emission signal. Ion currents were recorded using whole-cell patch clamp technique. Recording was made with an Axopatch 200B amplifier (Axon Instruments). Currents were filtered at 5 kHz and digitized by using Digidata 1321A interface. Data were interpolated using Origin6.0 (version 6.0, Microcal). Pipettes were pulled from borosilicate glass and had pipette resistances 1-4MΩ when filled with the intercellular solution. All recordings were made at room temperature (23±2° C.).

The intercellular solution contained 140 mM CsCl, 1 mM MgCl₂, 5 mM EDTA and 10 mM HEPES, pH 7.2. The extracellular solution contained 140 mM NaCl, 5 mM KC1, 1 mM MgCl₂, 2 mM CsCl₂ and 10 mM Glucose and 10 mM HEPES, pH 7.0-7.4. The extracellular solutions of pH less than 6.0 were buffered with 10 mM MES, but other constituents were identical. The rapid changes in extracellular pH were performed using Rapid Solution Changes (Bio-Logic Co.,).

Results

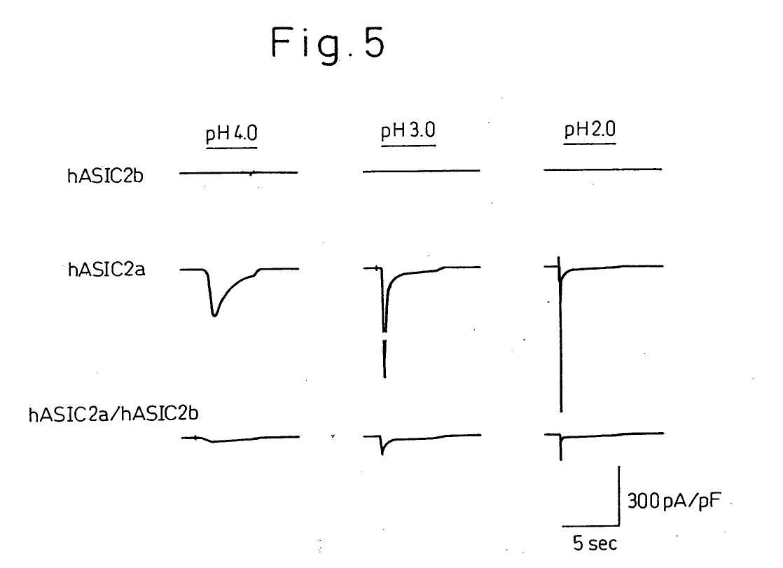

As can be seen from FIG. 5, hASIC2a and hASIC2b were expressed in CHO-K1 cells, and inward currents evoked by 5 sec application of low pH solution were recorded. In ASIC2a expressing cells, acid-induced inward currents were obtained at pH values (2.0-4.0) examined, however, no currents were obtained in ASIC2b expressing cells at any pH values. Thus, it was found that hASIC2b was inactive as an ion channels by itself. Next, hASIC2b was co-expressed with hASIC2a to see the effect on channel properties of hASIC2a. HASIC2a/hASIC2b co-expressing cells showed very small acid-sensing currents compared with hASIC2a expressing cells. These results suggest that hASIC2b exerts inhibitory effect on acid-induced ion currents.

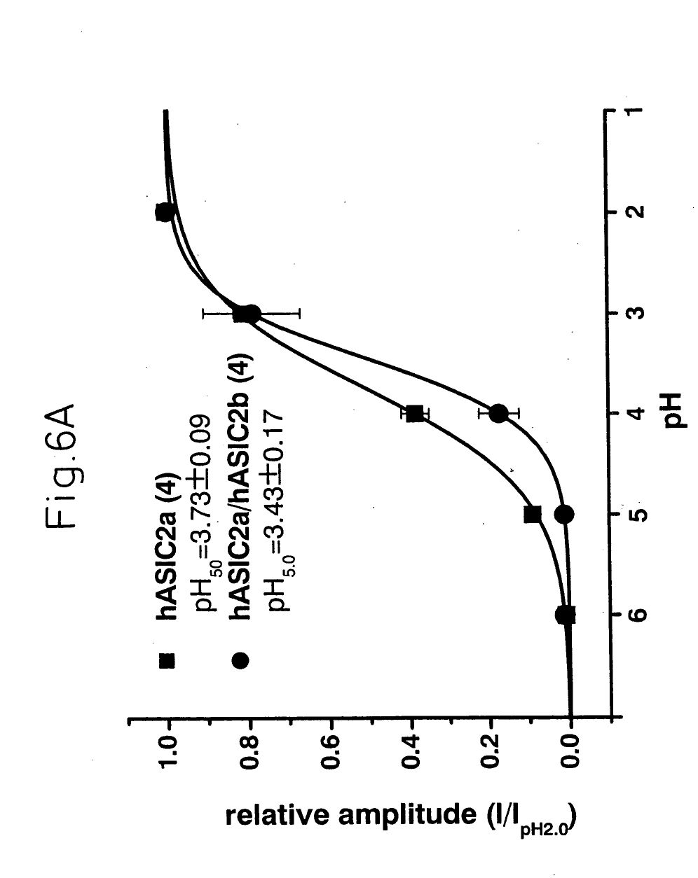

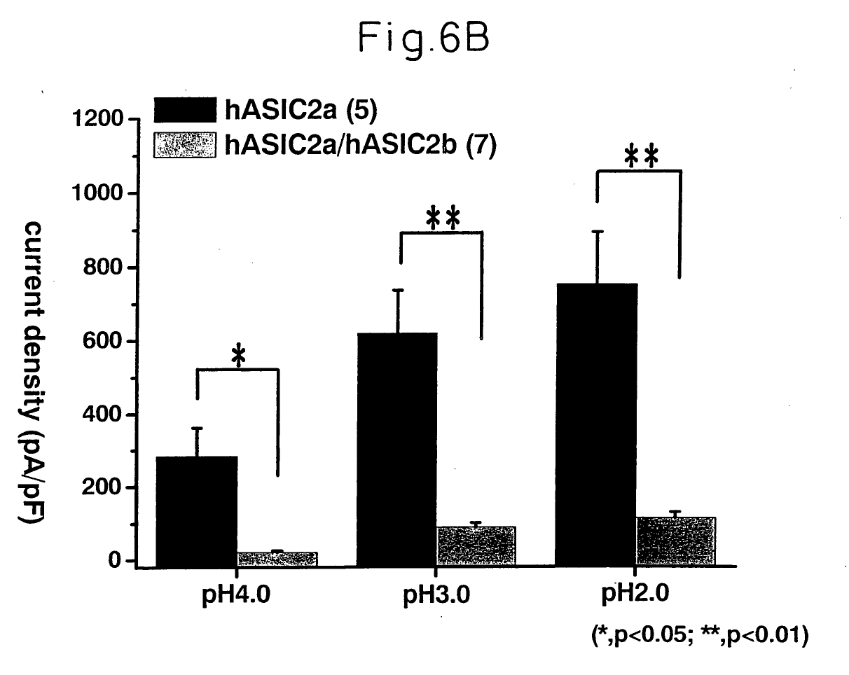

The pH dependence of the acid-sensing currents of hASIC2a and hASIC2a/hASIC2b were examined by decreasing extracellular pH. The pH₅₀ value for activation of hASIC2a and hASIC2a/hASIC2b were 3.73±0.09 (n=4) and 3.43±0.17 (n=4), respectively (FIG. 6A). There were no significant changes in sensitivity to acidic stimuli by co-expression of hASIC2a with hASIC2b. Next, as shown in FIG. 6B, peak current density was compared between hASIC2a and hASIC2a/hASIC2b transformants at pH2.0, 3.0, and pH4.0.(hASIC2a ; 752.6±140.6 pA/pF at pH2.0, 619.6±116.8 pA/pF at pH3.0, 284.4±77.7 pA/pF at pH4.0, hASIC2a/hASIC2b ; 112.8±16.3 pA/pF at pH2.0, 88.9±12.0 pA/pF at pH3.0, 21.0±4.83 pA/pF at pH4.0)

A series of studies performed here demonstrated the inhibitory role of hASIC2b in acid-induced currents generated by hASIC2a. That suggests a critical role of hASIC2b in regulating acid-induced currents in the health and disease conditions of human physiological systems.

All documents cited herein, including patents and patent applications, are hereby incorporated by reference.

It will be appreciated that the foregoing is provided by way of example only and modification of details may be made without departing from the scope of the invention.

1. A polynucleotide comprising one or more of:
(a) a polynucleotide encoding the polypeptide as set forth in SEQ ID NO:2;
(b) a polynucleotide comprising a nucleotide sequence of SEQ ID NO:1;
(c) a polynucleotide comprising a nucleotide sequence that has at least 70% identity to the polynucleotide of (a) or (b);
(d) a polynucleotide comprising a nucleotide sequence which is capable of hybridizing to the polynucleotide of any one of (a) to (c);
(e) a complement to the polynucleotide of any one of (a) to (d); or
(f) a polynucleotide fragment of the polynucleotide of any one of (a) to (e).

2. The polynucleotide according to claim 1, encoding a human acid sensing ion channel (ASIC) 2b.

3. A vector comprising the polynucleotide according to claim 1.

4. A host cell transformed or transfected with the vector according to claim 3.

5. Transcribed RNA product of the polynucleotide according to claim 1.

6. An RNA molecule or fragment thereof which is antisense in relation to the RNA product of claim 5 and is capable of hybridizing thereto.

7. A ribozyme or zinc finger protein capable of binding the polynucleotide according to claim 1.

8. A process of producing a polypeptide or fragment thereof comprising culturing the transformed/transfected host cell according to claim 4 under conditions sufficient for the expression of said polypeptide or fragment.

9. A process of producing cells capable of expressing a polypeptide or fragment thereof comprising transforming or transfecting cells with the vector according to claim 3.

10. Cells produced by the process according to claim 9.

11. A membrane preparation of the cells according to claim 10.

12. A polypeptide or a fragment thereof produced by the process according to claim 8.

13. A polypeptide or a fragment thereof produced by the process according to claim 9.

14. A polypeptide comprising:
(a) a polypeptide having the deduced amino acid sequence translated from the polynucleotide sequence in SEQ ID NO:1 or variants, fragments, homologues, analogues and derivatives thereof; or
(b) a polypeptide of SEQ ID NO:2 and variants, fragments, homologues, analogues or derivatives thereof.

15. The polypeptide according to claim 14 fused with an additional human acid sensing ion channel (hASIC) selected from the group consisting of hASIC1a, hASIC1b, hASIC2a, hASIC3, hAISC4, or their derivatives.

16. An antibody against the polypeptide according to claim 14.

17. A compound, which modulates the polypeptide according to claim 14.

18. A method of screening for substances capable of modulating the polypeptide according to claim 14, which comprises:
(a) contacting a substance to be tested with cells expressing at least one molecule of the polypeptide according to claim 14 and optionally at least one molecule of an additional human acid sensing ion channel (hASIC) selected from the group consisting of hASIC1a, hASIC1b, hASIC2a, hASIC3, hAISC4, or their derivatives on their surface;
(b) measuring the effects of the substance on the transport functions of said polypeptide or at least one of said hASICs or derivatives; and
(c) identifying the substances that have a positive or negative effect on the transport functions.

19. A method of identifying a compound, which binds to and modulates the polypeptide according to claim 14 comprising contacting said polypeptide with a candidate compound and determining whether modulation occurs.

20. The method according to claim 18, which comprises:
(a) contacting a compound with cells expressing at least one molecule of the polypeptide according to claim 14 and optionally at least one molecule of an additional human acid sensing ion channel (hASIC) selected from the group consisting of hASIC1a, hASIC1b, hASIC2a, hASIC3, hAISC4, or their derivatives on their surface, said polypeptide or at least one of said hASICs or derivatives being associated with a second component capable of providing a detectable signal in response to the binding of a compound to said polypeptide or at least one of said hASICs or derivatives; said contacting being under conditions sufficient to permit binding of compounds to the polypeptide or at least one of said hASICs or derivatives; and
(b) identifying a compound capable of binding the polypeptide or at least one of said hASICs or derivatives by detecting the signal produced by said second component.

21. A method for the treatment of a patient having a need to modulate the polypeptide according to claim 14 comprising administering to the patient a Therapeutically effective amount of the compound according to claim 17.

22. The method according to claim 21, wherein said compound is a polypeptide and a therapeutically effective amount of the compound is administered by providing to the patient DNA encoding said compound and expressing said compound in vivo.

23. Use of the antibody according to claim 16 in the manufacture of a medicament for the treatment of a patient having a need to modulate the polypeptide according to claim 14.

24. A method for the treatment of a patient having need to modulate the polypeptide according to claim 14, comprising administering to the patient a therapeutically effective amount of the antibody according to claim 16.

25. Cells genetically engineered ex vivo or in vivo to express, overexpress, underexpress or to exhibit targeted insertion or deletion of the polypeptide according to claim 14.

26. A transgenic non-human animal comprising cells according to claim 25.
